(19) United States
(12) Patent Application Publication
(10) Pub. No.: US 2004/0023784 A1
(43) Pub. Date: Feb. 5, 2004

(54) Bioactive biphasic ceramic compositions for artificial bone and method for making the same

(76) Inventors: Hyun Seung Yu, Seoul (KR); Kug Sun Hong, Seoul (KR); Choon Ki Lee, Seoul (KR); Dong Ho Lee, Seoul (KR); Jae Hyup Lee, Seoul (KR); Bong Soon Chang, Seoul (KR); Su Jin Kim, Seoul (KR); Sung Soo Chung, Kyoungki-do (KR)

Correspondence Address: ROSENBERG, KLEIN & LEE, 3458 ELLICOTT CENTER DRIVE-SUITE 101, ELLICOTT CITY, MD, 21043, US

(21) Appl. No.: 10/357,219

(22) Filed: Feb. 4, 2003

(30) Foreign Application Priority Data
Jul. 30, 2002 (KR) 2002-44845

(51) Int. Cl. A61F 2/28; C04B 35/00

(52) U.S. Cl. 501/1; 106/35; 623/23.56

(57) ABSTRACT

A bioactive biphasic ceramic composition combining apatite and wollastonite is disclosed, in order to solve the defect of apatite ceramic that has poor bioactivity though it is excellent in biocompatibility, which has improved bioactivity, as compared to monophasic ceramics of apatite or wollastonite. The ceramic composition is produced by steps of: providing a composition including powders of apatite of formula Ca₁₀(PO₄)₆X, in which X is any one of O, (OH)₂, CO₃, F₂ and Cl₂, and wollastonite (CaSiO₃) in a weight ratio of 5:95 to 90:10, forming the composition into a desired body by press or forming the composition into a porous body, and sintering the formed body.

### BACKGROUND OF THE INVENTION

1. Field of the Invention

The present invention relates to a bioactive biphasic ceramic composition for artificial bone and a method for making the same. More particularly, the present invention relates to a bioactive biphasic ceramic composition combining apatite and wollastonite, in order to solve the defect of apatite ceramic that has poor bioactivity despite excellent biocompatibility, which has improved bioactivity, as compared to apatite ceramics or wollastonite ceramics, and a method for producing the same.

2. Background of the Related Art

In general, materials for artificial bone should have an ability to directly bind to bone. Particularly, for rapid bone fusion, they should have a high affinity for bone tissue and be able to chemically bind to bone. A representative example of such materials is bioactive ceramics. The bioactive ceramics can directly bind to a bone, unlike other polymers and metals. For example, the bioactive ceramics include calcium phosphate ceramics such as hydroxyapatite and bioactive glass, termed Bioglass®.

Hydroxyapatite (HA: Ca₁₀(PO₄)₆(OH)₂) is a compound comprising the same elements (calcium, phosphorus) with inorganic substances making up bone of our bodies and also has chemical properties most similar to them. Also, tricalcium phosphate (TCP: Ca₃(PO₄)₂) and calcium pyrophosphate (CCP: Ca₂P₂O₇) having a ratio of calcium to phosphorus lower than that of hydroxyapatite can be directly bound to bone.

Meanwhile, bioactive glass was known by Hench of USA who reported Bioglass® of specific compositions capable of chemically binding to bone. The glass of compositions comprises mainly soda (Na₂O), silica (SiO₂) and calcium oxide (CaO). Hench disclosed the bioactive glass compositions in U.S. Pat. Nos. 4,103,002, 4,171,544, 4,234,972, 4,851,046, 4,775,646, 5,074,916, 5,840,290 and 5,981,412. Since these glasses of compositions have a bioactivity level higher than those of calcium phosphate ceramics including hydroxyapatite, they are expected to bind to bone in a short time. Furthermore, some of them have such a high bioactivity level according to their compositions that they can even bind to soft tissue. However, the bioactive glass has significantly poor mechanical strength due to the intrinsic property of glass and thus, has a limitation in its application to artificial bone. Therefore, there have been conducted intensive researches to solve this problem.

Kokubo et al. of Japan developed Cerabone-AW which is produced by crystallizing of a glass composition comprising 44.7 weight parts of CaO, 34.0 weight parts of SiO₂, 6.2 weight parts of P₂O₅, 0.5 weight parts of CaF₂ and 4.6 weight parts of MgO and has an improved mechanical strength while having a high bioactivity, on 1982 (Kokubo et al., Bull. Inst. Chem. Res., Kyoto Univ., 60 (1982), pp.260-268). Kokubo et al. disclosed the bioactive glass-ceramics compositions in Japanese Patent Laid-Open Publication Nos. 57-191252, 61-091041, 3-131263 and 3-272771.

The high bioactivity of bioactive glass or glass-ceramics, compared to calcium phosphate ceramics including hydroxyapatite are attributable to a surface reaction with body fluid. When the interface of the bioactive glass or glass-ceramics binding to bone was observed, for example by an electron microscope, there is shown a thin layer comprising calcium and phosphorus between bone and a implant which has been clarified as hydroxycarbonate apatite layer (HCA layer) having chemical properties similar to inorganic ingredients of bone and has been found to provide a site favorable to attachment and growth of bone cells and formation of bone tissue.

This layer is formed by interaction between body fluid and glass or glass-ceramics according to a mechanism, by which calcium contained in the glass ingredients is extracted from the surface and silica on the surface reacts with water to form silanol (Si—OH) group. It is known that the silanol group provides a nuclei forming site where hydroxycarbonate apatite can be crystallized and the extracted calcium functions to increase supersaturation of body fluid to hydroxycarbonate apatite, whereby the layer of hydroxycarbonate apatite can be readily formed.

On the contrary, the calcium phosphate ceramics do not contain silica in the constituent ingredients and thus, cannot produce a hydroxycarbonate apatite layer through a reaction with body fluid. For the calcium phosphate ceramics, dissolution/recrystallization occurs on the surface by the action of surrounding cells after grafting, whereby the surface is modified to be analogous to hydroxycarbonate apatite which is similar to inorganic substances of bone. The surface modification by cells is slower than that of the modification by the reaction with body fluid and consequently, the calcium phosphate ceramics show a low bioactivity.

However, the bioactive glass and the glass-ceramics are produced through more complex process, as compared to the calcium phosphate ceramics. The calcium phosphate ceramics are produced by 3-steps of mixing-calcination-sintering while the bioactive glass requires at least 4-steps of mixing-melting-quenching/forming-annealing and the glass-ceramics requires at least 4-steps of mixing-melting-quenching-crystallization. Also, in performing a process for producing glass, there are several difficulties that mixed powders should be melted completely at a high temperature of at least 1450° C., and the melt of the high temperature then should be immediately quenched. Further, for glass-ceramics, glass bulk should be pulverized. However, the pulverization of glass to several microns (μm) cannot be accomplished by a method commonly used to pulverize ceramics such as ball-mill since glass has a high hardness.

In general, material with superior bioactivity should be used for more rapid bone fusion. According to techniques up to date, glass mainly constituting of calcium oxide and silica suits to the above purpose. However, the process for producing glass is complex and includes operations at a considerably high temperature of at least 1450° C., causing increase in process cost. Also, it is difficult to maintain and repair equipments for the process.

### SUMMARY OF THE INVENTION

Thus, in order to solve the problems involved in the prior arts, it is an object of the present invention to provide a bioactive biphasic ceramic composition for artificial bone which has excellent bioactivity comparable to the existing bioactive glass and glass-ceramics and can be simply produced through a known ceramic processing at a relatively low temperature and a method for making the same.

To achieve the above object, in one embodiment, the present invention provides a bioactive biphasic ceramic composition for artificial bone comprising an apatite of formula Ca₁₀(PO₄)₆X, in which X is any one of O, (OH)₂, CO₃, F₂ and Cl₂, and a wollastonite (CaSiO₃) in a weight ratio of 5:95 to 90:10.

In another aspect, the present invention provides a method for producing a bioactive biphasic ceramic composition for artificial bone comprising the steps of:

preparing a composition comprising powders of an apatite of formula Ca₁₀(PO₄)₆X, in which X is any one of O, (OH)₂, CO₃, F₂ and Cl₂, and a wollastonite (CaSiO₃) in a weight ratio of 5:95 to 90:10,

forming the composition into a desired body by press or forming the composition into a porous body, and

sintering the formed body at a temperature of 1,200 to 1,400° C.

### BRIEF DESCRIPTION OF THE DRAWINGS

The above and other objects, features and advantages of the present invention will be apparent from the following detailed description of the preferred embodiments of the invention in conjunction with the accompanying drawing, in which:

FIG. 1 is a graph illustrating sintering properties of the ceramics combining apatite and wollastonite;

FIGS. 2a to 2f are photographs illustrating surfaces of respective specimens, taken by an electron microscope to confirm whether an hydroxycarbonate apatite layer has been produced after soaking in simulated body fluid for 1 day; and

FIGS. 3a to 3e are photographs illustrating microstructure of specimens which have been sintered for 2 hours at 1300° C., taken by a scanning electron microscope.

### DETAILED DESCRIPTION OF THE PREFERRED EMBODIMENT

Now, the present invention is described in detail.

The bioactive biphasic ceramic composition for artificial bone can be produced by a known ceramic processing. Therefore, its production process is simple and its process temperature is as relatively low as 1,200 to 1,400° C.

The wollastonite (CaSiO₃) is a ceramic synthesized from calcium dioxide and silica in a molar ratio of 1:1 and is practically known to have bioactivity, though bioactivity of its own is not yet known clearly. It is generally considered that its bioactivity is inferior to those of bioactive glass and crystallized glass.

The wollastonite has two polymorphs; α phase and β phase. The β-wollastonite is a low temperature phase and is transformed into the α-wollastonite which is a high temperature phase at a temperature of around 1120° C. The phase transition from β- to α-phase is irreversible. That is, once the β phase is transited to the α phase, it never returns back to the β phase. In terms of bioactivity, it is known that the α-wollastonite is superior to the β-wollastonite. It is believed that this is because the α phase has a much higher solubility than the β phase, and therefore increases supersaturation of calcium in body fluid and forms the silanol group in a more amount.

The present inventors has discovered that when the apatite with low bioactivity is combined with the wollastonite which has higher bioactivity than apatite, but lower than conventional bioactive glass, the resultant composite shows bioactivity comparable to the bioactive glass and completed this invention based on the discovery.

According to the present invention, the mixing ratio (w/w) of apatite to wollastonite is 5:95 to 90:10, preferably 20:80 to 80:20. When the mixing ratio of apatite to wollastonite is less than 5:95(w/w), the resultant composite is mainly composed of the wollastonite and an effect of the apatite is insignificant. Therefore, the composite shows bioactivity similar to a single ceramic of wollastonite. In an in-vitro test by a simulated body fluid soaking experiment, it was observed that a hydroxycarbonate apatite layer fail to cover the whole surface of a specimen. When the ratio is greater than 90:10, the resultant composite shows low bioactivity since the content of apatite with poor bioactivity is high. In the simulated body fluid soaking experiment, it was observed that no hydroxycarbonate apatite layer was formed even after soaking in simulated body fluid for 20 days.

Also, the composite after forming is preferably sintered at a temperature of 1,200 to 1,400° C. When the formed body is sintered at a temperature of less than 1,200° C., sintering is not performed sufficiently. Therefore, the resultant sintered body has a relative density of 70% or less, and hence shows a very low mechanical strength. On the other hand, when the formed body is sintered at a temperature exceeding 1,400° C., it reaches the melting point (1410° C.), and therefore the specimen melts.

In terms of bioactivity, the effect of addition of the apatite in a small amount to the wollastonite is much greater than the effect of addition of wollastonite in a small amount to apatite. This is because the wollastonite is more soluble in body fluid, as compared to the apatite. The wollastonite provides calcium and silanol group needed to produce the hydroxycarbonate apatite layer and phosphorus contained in the apatite additionally provides cites needed to produce the hydroxycarbonate apatite layer. Accordingly, composite ceramics of a small amount of apatite with wollastonite shows much more improved bioactivity.

Now, the method for producing the bioactive biphasic ceramic composition for artificial bone according to the present invention will be described in detail.

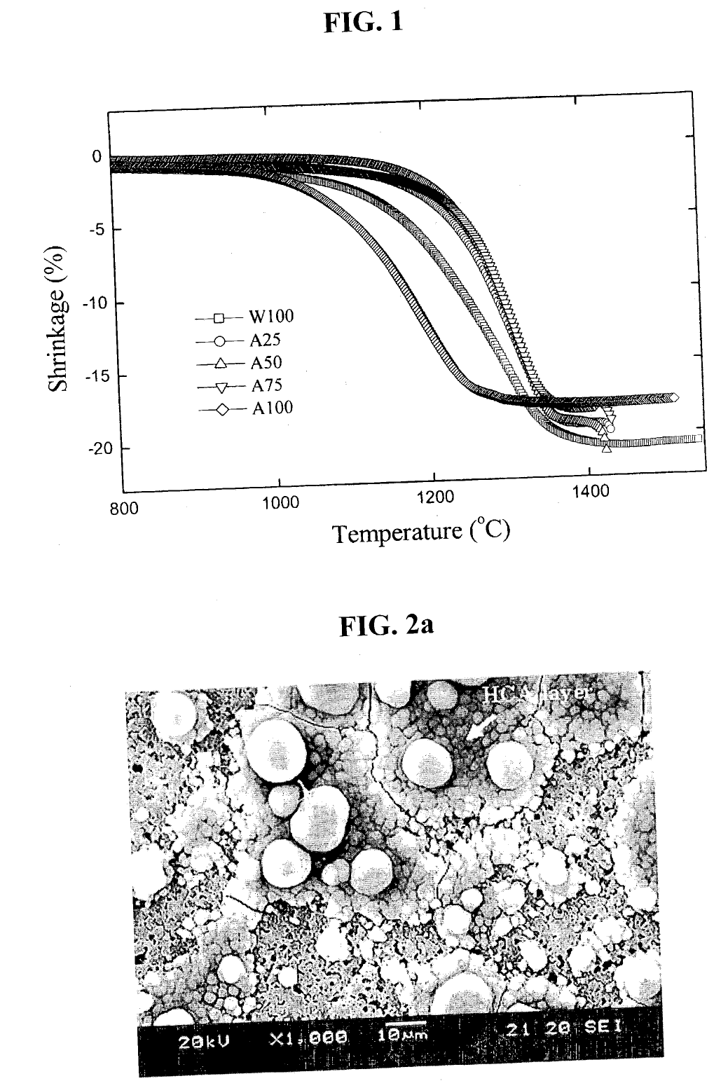

In the first aspect of the present invention, a bioactive biphasic ceramic composition combining apatite and wollastonite in a specific ratio is provided. The bioactive biphasic ceramic composition according to the present invention is prepared by separately synthesizing apatite and wollastonite, followed by preliminary pulverizing and uniformly mixing the pulverized apatite and wollastonite in a specific ratio. Here, the mixing ratio of apatite and wollastonite is 5:95 to 90:10 (w/w), preferably 20:80 to 80:20. The powder mixture of apatite and wollastonite is press-formed to produce a formed body, which is then minutely sintered from a starting temperature of 1,200° C. and a ending temperature of 1,400° C., as shown in FIG. 1.

Meanwhile, it was shown that a ceramic composed of only the apatite does not produce the hydroxycarbonate apatite layer on the surface in a simulated body fluid soaking experiment even after 2 months due to low bioactivity. Also, a ceramic composed of only the wollastonite has a high solubility in body, and thereby low in vivo stability. It was shown that in a simulated body fluid soaking experiment of the ceramic of wollastonite, a hydroxycarbonate apatite layer does not cover the entire fluid contact surface.

However, a composite of the two ceramics can produce a hydroxycarbonate apatite layer covering the entire fluid contact surface in a short period of time. Also, its microstructure has a particle size smaller than the single ceramic, whereby it is possible to expect an increased mechanical strength as the particle size decreases.

It is believed that the reason why the composite ceramic of apatite and wollastonite has an increased bioactivity, as compared to the monophasic ceramics is because the wollastonite (CaSiO₃) has a high solubility, the dissolved wollastonite increases the supersaturation of calcium in simulated body fluid and silica of wollastonite and phosphate group of apatite (PO₄³⁻) can provide together the favorable sites where a nuclei of the hydroxycarbonate apatite can be formed. Therefore, the composite according to the present invention can have a bioactivity comparable to that of bioactive glass or glass-ceramics. Also, since the wollastonite and apatite are much alike in sintering properties, the ceramic composition comprising them can be advantageously well sintered to produce a dense ceramic.

As described above, the bioactive biphasic ceramic produced according to the present invention shows the bioactivity which is not inferior to existing bioactive glass and glass-ceramics, in a simulated body fluid soaking experiment but is greatly improved, as compared to the apatite.

### EXAMPLES

Now, the present invention is described in further detail using the following examples. However, it should be understood that the present invention is not limited thereto.

### Examples 1 to 6, Comparative Example 1, and Prior Arts 1 and 2

Calcium carbonate (99.99%) and calcium pyrophosphate (99.9%) were mixed in a molar ratio of total calcium to phosphorus of 1.667 and the mixture was calcined at 1150° C. for 12 hours to synthesize apatite. Also, calcium carbonate (99.99%) and silica (99.9%) was mixed in a molar ratio of total calcium to silica of 1 and the mixture was calcined at 1300° C. for 4 hours to synthesize wollastonite.

These synthesized powders were weighed according to the ratio for Examples 1 to 6 and Comparative Example 1 described in Table 1 and mixed and pulverized by a ball-mill with ZrO₂ media for 24 hours. The resulting powder mixture was then press-formed at a hydrostatic pressure of 1000 kg/cm², to obtain a disc-shaped specimen having a diameter of 8 mm and a thickness of 3 mm.

The specimens of Examples 1 to 6 according to the present invention, Comparative Example 1 and single phase specimens composed of apatite and wollastonite of Prior art Examples 1 and 2 were sintered at 1200 to 1350° C. for 2 hours. Here, the temperature was elevated during sintering at 5° C./min. After completion of sintering the samples was furnace-cooled. The sintered specimens were examined by phase analysis, bulk density measurement, bioactivity evaluation according to the following methods and the results are shown in Table 1.

(1) Phase Analysis

The formed body of each ceramic composition after sintering was examined by X-ray diffraction to confirm the produced phase. The measurement was performed on an area of 2θ 20 to 40° at a scanning speed of 0.02°/0.5 seconds.

(2) Bulk Density

The bulk density of the sintered body of each composition was measured by the Archimedes' method and the value of the bulk density was divided by a value of theoretical density to obtain a relative density.

(3) Bioactivity Evaluation

35 cc of simulated body fluid (SBF) containing inorganic substances similar to human blood plasma was poured to a polyethylene bottle and two specimens having a diameter of 8 mm and a thickness of 2 mm were placed therein. The bottle was stored in a chamber kept at 36.5° C. for a predetermined period of time, then washed with distilled water and acetone. The resulting specimen was examined for their surfaces under an electron microscope and subjected to the X-ray diffraction analysis. In general, as a hydroxycarbonate apatite layer is quickly formed over the entire surface of the specimen, the bioactivity of the specimen is high.

TABLE 1

| | | Mixing | | | | Formation |
|---|---|---|---|---|---|---|
| | | ratio | | | Max. | of |
| Example | | (w/w) | | Sinterable | relative | hydroxycarbo |
| No. | Title | A* | B* | temp. | density | nate apatite |
| Prior | A100 | 100 | 0 | 1250, | 97% | No formation |
| art 1 | | | | 1300° C. | | until 30 |
| | | | | | | days |
| Prior | W100 | 0 | 100 | 1300° C. | 98% | Formed after |
| art 2 | | | | | | 1 day, |
| | | | | | | but on parts |
| | | | | | | of the |
| | | | | | | surface |
| Example | A5 | 5 | 95 | 1300° C. | 97% | Formed after |
| 1 | | | | | | 1 day, but |
| | | | | | | complete |
| | | | | | | formation on |
| | | | | | | the entire |
| | | | | | | surface |
| | | | | | | after 10 |
| | | | | | | days |
| Example | A10 | 10 | 90 | 1300° C. | 98% | Formed after |
| 2 | | | | | | 1 day, but |
| | | | | | | complete |
| | | | | | | formation on |
| | | | | | | the entire |
| | | | | | | surface |
| | | | | | | after 7 |
| | | | | | | days |
| Example | A25 | 25 | 75 | 1300° C. | 98% | Formed after |
| 3 | | | | | | 1 days on |
| | | | | | | the entire |
| | | | | | | surface |
| Example | A50 | 50 | 50 | 1300° C. | 97% | Formed after |
| 4 | | | | | | 1 day, on |
| | | | | | | the entire |
| | | | | | | surface |
| Example | A75 | 75 | 25 | 1300° C. | 98% | Formed after |
| 5 | | | | | | 10 days, on |
| | | | | | | the entire |
| | | | | | | surface |
| Example | A90 | 90 | 10 | 1300° C. | 97% | Formed after |
| 6 | | | | | | 25 days, on |
| | | | | | | the entire |
| | | | | | | surface |
| Comp, | A95 | 95 | 5 | 1300° C. | 97% | No formation |
| Example | | | | | | until 60 |
| 1 | | | | | | days |

*A: apatite, B wollastonite (casiO₃)

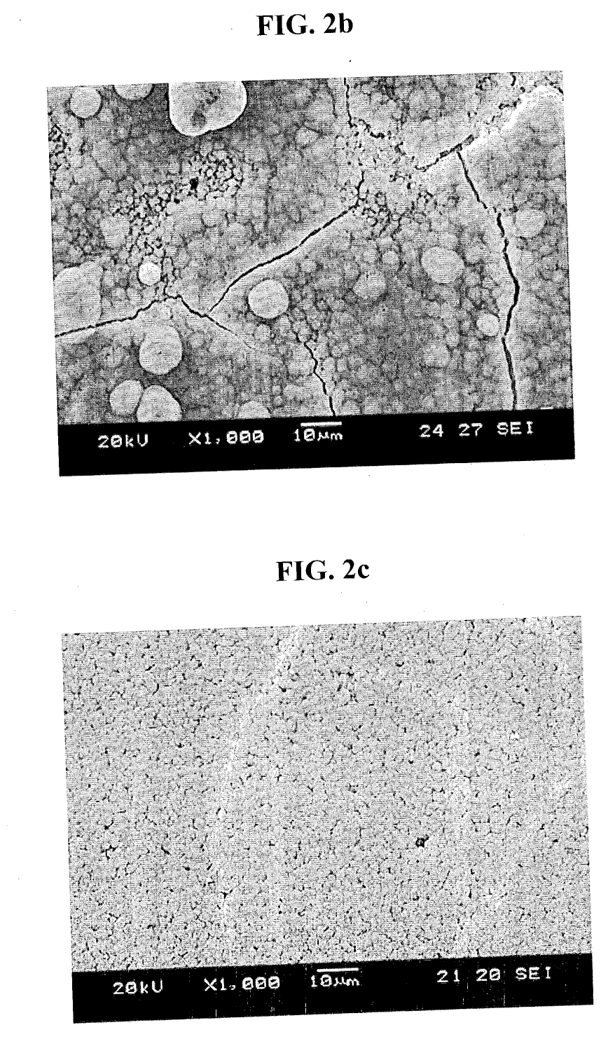

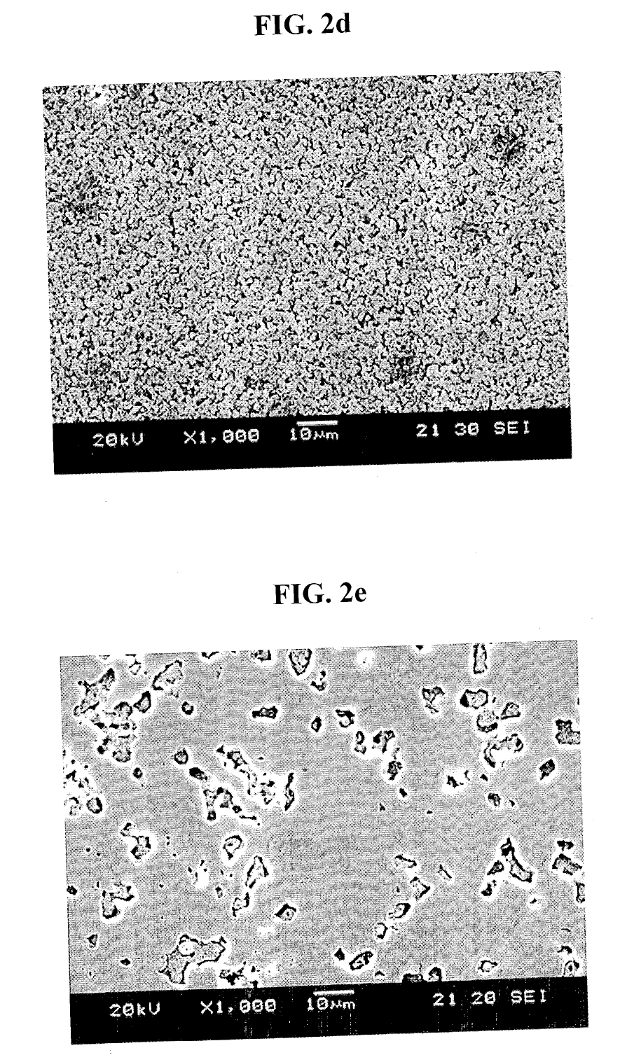

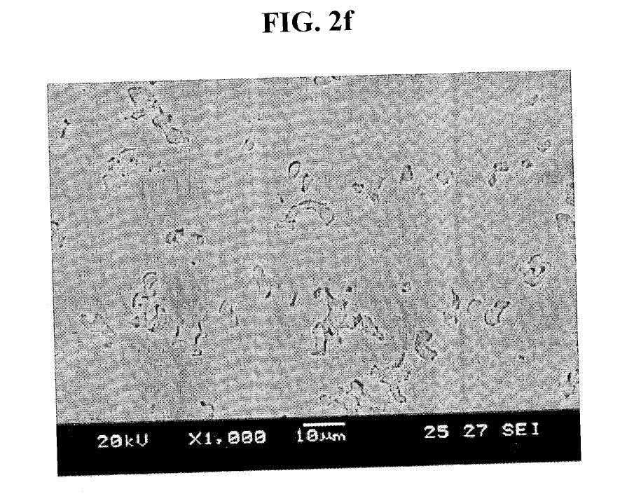

FIG. 1 is a graph illustrating sintering properties of the ceramics combining apatite and wollastonite and FIGS. 2a to 2f are SEM photographs of surfaces of respective specimens to confirm whether an hydroxycarbonate apatite layer has been produced after soaking in simulated body fluid for 1 day.

As can be seen from Table 1 and FIGS. 2a to 2f, in the ceramic composed of apatite alone of Comparative example 1, no formation of hydroxycarbonate apatite was observed until 60 days after soaking in simulated body fluid. In the ceramic composed of wollastonite alone of Prior art 2, formation of hydroxycarbonate apatite was observed after 1 day. The hydroxycarbonate apatite did not cover the entire surface, but formed sporadically (FIGS. 2a and 2b). It was noted that as the content of apatite increased, the time taken for formation of the hydroxycarbonate apatite layer on the entire surface was reduced and a uniform layer could be obtained (FIGS. 2c and 2d). However, when the content of apatite exceeded 50%, the formation of the hydroxycarbonate apatite layer slowed down and there were again observed spots where the hydroxycarbonate apatite layer was not formed (FIGS. 2e and 2f).

Consequently, as seen from the results of Table 1, when the mixing ratio of apatite to wollastonite was 5:95 to 90:10, the bioactivities of the produced ceramics were improved. Particularly, it was noted that composite ceramics of the mixing ratio of 20:80 to 80:20 showed bioactivities comparable to conventional bioactive glass and glass-ceramics.

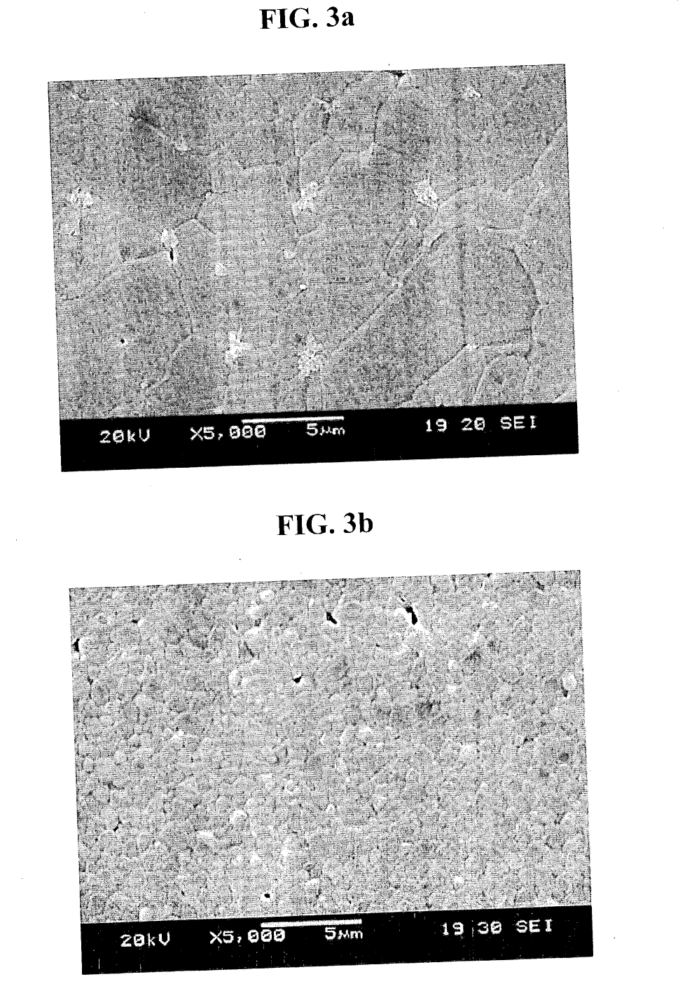

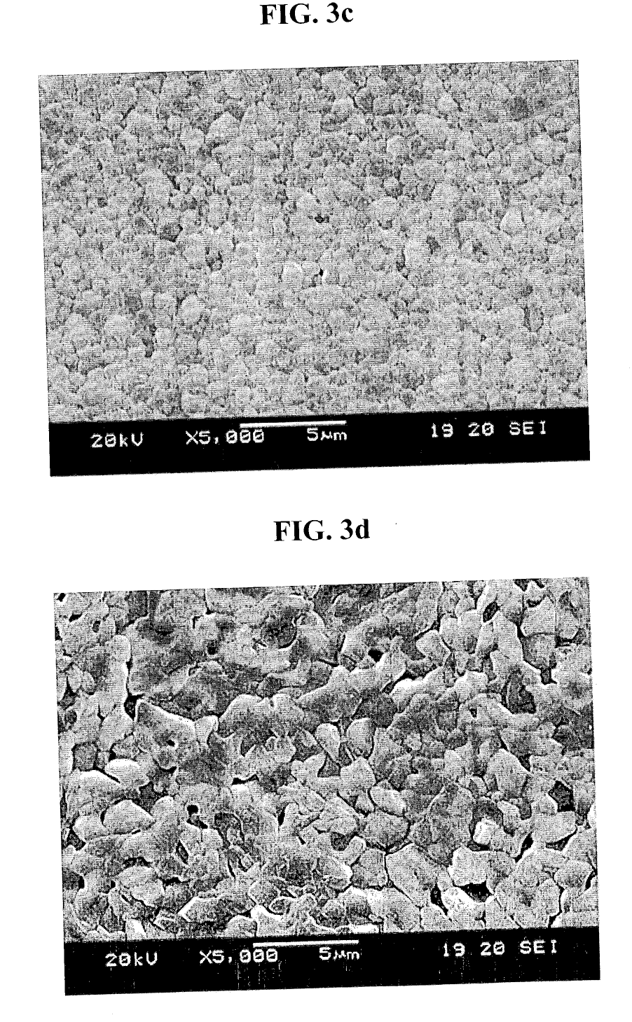

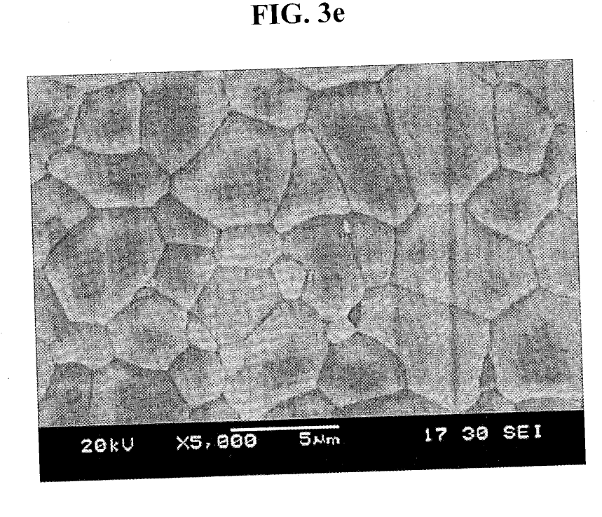

Since material for artificial bone is required to have a certain mechanical strength level, the ceramics prepared from the above examples were examined for their microstructures (FIGS. 3a to 3e, photographs of microstructure of specimens which has been sintered for 2 hours at 1300° C., taken by a scanning electron microscope). The wollastonite ceramics had abnormal grain growth due to liquid phase sintering, but the apatite ceramics showed to have a large grain size due to grain growth. On the contrary, the biphasic apatite/wollastonite ceramics had microstructures of grains having a grain size of about 1 μm without abnormal grain growth. The ceramics formed of finely small grains generally can have a high mechanical strength since they have a great resistance against crack propagation. Therefore, it is noted that the ceramics of Examples 1 to 6 according to present invention have advantageous microstructures in terms of mechanical strength.

As described above, the present invention can very simply and economically produce artificial bone having a bioactivity comparable to those of the existing bioactive glass and glass-ceramics. Therefore, it can be very advantageous to produce artificial bone for rapid bone fusion.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

What is claimed is:

1. A method for producing a bioactive biphasic ceramic composition for artificial bone comprising the steps of:
preparing a composition comprising powders of an apatite of formula Ca₁₀(PO4)₆X, in which X is any one of O, (OH)₂, CO₃, F₂ and Cl₂, and a wollastonite (CaSiO₃) in a weight ratio of 5:95 to 90:10,
forming the composition into a desired body by press or forming the composition into a porous body, and sintering the formed body.

2. The method according to claim 1, wherein the step of sintering is performed at a sintering temperature of 1,200 to 1,400° C.

3. A bioactive biphasic ceramic composition for artificial bone comprising an apatite and a wollastonite in a weight ratio of 5:95 to 90:10.

4. The bioactive biphasic ceramic composition according to claim 3, wherein the composition comprises apatite and wollastonite in a weight ratio of 20:80 to 80:20.
